(19) United States
(12) Patent Application Publication
(10) Pub. No.: US 2024/0058148 A1
(43) Pub. Date: Feb. 22, 2024

(54) SYSTEMS AND METHODS FOR REMODELING AN EAR

(71) Applicant: EarGear, LLC, Broiklyn, NY (US)

(72) Inventor: Gila R. Weinstein, Brooklyn, NY (US)

(21) Appl. No.: 18/235,335

(22) Filed: Aug. 17, 2023

Related U.S. Application Data
(60) Provisional application No. 63/398,590, filed on Aug. 17, 2022.

(51) Int. Cl. A61F 5/01

(52) U.S. Cl. CPC A61F 5/01

(57) ABSTRACT

Non-surgical correcting apparatuses for a deformed external ear are provided. The correcting apparatuses may be worn externally to correct a deformity or malformation on the external ear through remodeling or reshaping over a period of time. The correcting apparatuses may be secured in place with associated attachment components coupled to the correcting components. The correcting apparatuses are configured to address the helix, helical rim, antihelix, conchal bowl, ear lobe, and triangular fossa of the ear.

### CROSS-REFERENCE TO RELATED APPLICATIONS

This application claims benefit of priority to U.S. Provisional No. 63/398,590 filed Aug. 17, 2022, the entire contents of which are incorporated herein by reference.

### FIELD

The present disclosure relates to systems and methods for remodeling portions of the external ear, and more particularly, to non-surgical correcting apparatuses for correcting a deformity or malformation on the external ear through remodeling or reshaping over a period of time.

### BACKGROUND

The external human ear is an important anatomical structure that aids in hearing by directing sound waves down the auditory canal. In some newborns, the external ear can be deformed. When infants are born with an irregular ear shape, they are said to have a congenital auricular deformity. This can range from an auricular deformity with no missing tissue (an abnormally shaped ear), to a malformation (missing tissue), to microtia (the external ears are not fully developed).

Various areas of the ear can be deformed, malformed or missing entirely, either at birth or after injury. Common ear deformities include a Stahls deformity (often referred to as a Spock ear), ear prominence, helical rim irregularities, constricted ear, cup ear, a lop ear (i.e., the helical rim is folded over, covering a portion of the antihelix) or a cryptotia, whereby a portion of the top of the ear is under the temporal scalp skin. An external ear deformity can cause a range of issues from cosmetic abnormalities to hearing and functional problems. Functional problems may include a variety of complications, such as difficulty wearing a hearing aid when there is a blocked ear canal. Only a small percentage of such deformities self-correct within the first week or two of life. Following the neonatal stage, an ear deformity will often require surgery to correct.

External ear shaping and/or protection mechanisms are known. For example, U.S. Pat. No. 7,850,702 pertains to a clamp in the form of a mainly U-shaped or V-shaped device that serves to non-invasively affect a cartilage fold on, for example, the exterior ear, by exerting a stretching and compressive force. U.S. Pat. No. 9,023,105 pertains to a system and method for correcting misshaped ears using a molding device having one or more braces supporting a scaphal mold. U.S. Patent Application Publication No. 2012/0124719 pertains to methods and apparatuses for protecting the external ear from the elements, such as heat, cold, wind, rain and/or snow. U.S. Patent Application Publication No. 2013/0068238 pertains to an ear protector and an ear protector wrap. In some embodiments, the ear protector comprises: an inner wall, an outer wall, and a curved joining wall. International Patent Application Publication No. WO 2014/167381 pertains to a definitive preformed expander implant for ear reconstruction, using a reconstruction method which involves expanding autologous (the patient's own) tissues, and ideally performed in one surgical operation.

As noted, various systems and methodologies for correcting external ear deformities are known in the art. However, their structure and means of operation are substantially different from the systems and methods disclosed herein. These conventional devices fail to solve all the problems that are overcome by the present systems and methods for their use.

### SUMMARY

Systems for remodeling portions of the external ear and methods for their use are described herein. These remodeling and reshaping systems provide an array of uniquely configured wearable devices for treating specific areas of the external ear. These wearable devices or correcting apparatuses may be shaped and configured for remodeling or reshaping a distinct region or anatomical location of the external ear, and may be used alone, or in combination with one another, as part of a comprehensive system to treat the ear.

More particularly, non-surgical correcting apparatuses are provided for correcting a deformity or malformation on the external ear through remodeling or reshaping over a period of time. Each of these correcting apparatuses may be configured for use with an external ear of a newborn or young infant, though the apparatus may be equally effective for use on older children or adults. Using a typical infant's ear as a guide, the correcting apparatus is configured to remodel and/or reshape the deformed ear and correct its present deformities.

In one aspect, a correcting apparatus for remodeling a portion of an external ear can comprise a correcting component having a main body configured for positioning between a surface of the external ear and a helix of the external ear. The correcting component may be used with an attachment component that couples to the correcting component for maintaining the correcting component in place. The attachment component may be configured for placement around the helix for securing at least a portion of the correcting component against the helix. The main body may be shaped to conform to an inner surface of the helix to mold, reshape and/or remodel the helix and/or the helical rim of the ear.

In use, the correcting component may be placed within, or against, the deformed helix of a newborn or young infant for a time period sufficient to allow the deformed ear to remodel. The main body may be provided with a preformed shape corresponding to a desired shape of a normal helix of a human ear. Alternatively, the main body may be moldable or bendable, such that the main body may be molded or shaped during implementation.

The main body may have a substantially cylindrical shape with a circular cross-section, or it may have other suitable cross-sections (e.g., oval, rectangular, square or the like). In certain embodiments, the main body of the correcting component may have a central portion and first and second ends. The central portion may have an outer dimension larger than an outer dimension of the first and second ends. The first and second ends may have substantially the same outer dimension, or one of the ends may be larger than the other. In some embodiments, the first and second ends may have a different shape.

In certain embodiments, the attachment component may comprise a main body for coupling to at least a portion of the correcting component and one or more elongate projections or fingers extending from the main body. The main body may be elongate, with one or more fingers extending posteriorly. The posterior fingers are preferably spaced from each other and aligned with the main body such that they provide sufficient support to maintain the correcting component within the helix of the ear. The posterior fingers preferably have a length selected such that they may wrap around the helix to the rear surface of the ear to secure the correcting component against the helix. In certain embodiments, the attachment component may comprise two or more posterior fingers, or any suitable number of fingers in order to secure the correcting component in place (i.e., preferably around 2 to 10 fingers, more preferably between about 3 to 6).

The attachment component may further comprise one or more elongate projections or fingers extending anteriorly from the main body and configured to contact a surface of the external ear anterior to the helix, such as the antihelix, cymba, cavum, tragus or another adjacent surface. The anterior elongate member(s) function to further secure the correcting component to the helix.

The attachment component may comprise any suitable adhesive device, such as adhesive film, layer or tape, such as surgical tape, Velcro or hook and loop materials, temporary glue and other such known skin attachment mechanisms. In one embodiment, the attachment components may comprise a one-sided or double-sided surgical tape that is configured for adherence to the outer skin. The surgical tape may be configured to temporarily secure the correcting component to the external ear, while allowing for atraumatic removal after use.

In certain embodiments, the correcting component may comprise a relatively atraumatic outer portion for contacting the ear and an inner portion embedded within the outer portion for maintaining a shape of the outer portion. The inner portion may be malleable or bendable, such that the correcting component can be molded to generally conform to the helix, while having sufficient rigidity to remain in place during a time period of use sufficient to correct the deformity. The outer portion may comprise any suitable soft, atraumatic material for placement against the skin of an infant, such as silicone, polymer, plastic, fabric, foam or a blend thereof. The inner portion may comprise any suitable malleable or bendable, but substantially rigid, material, such as a metal or rigid polymer wire, flexible thread or rod, wire mesh or other bendable structure such as a sheet metal strip that can hold position, and the like.

In another aspect, a correcting apparatus for remodeling a portion of an external ear may comprise a correcting component for creating an antihelix. The correcting apparatus may comprise an elongate dome atop a panel, which may also be elongate, for attachment to the scalp skin behind the external ear where the antihelix is desired. The apparatus may be coupled to an attachment component such as an adhesive film, layer or tape, such as surgical tape, Velcro or hook and loop materials, temporary glue and other such known skin attachment mechanisms. This attachment component enables the correcting apparatus to be secured in position behind the external ear for a duration of time sufficient to create the antihelix.

In still another aspect, a correcting apparatus for remodeling a portion of an external ear may be configured as a constriction sheath. The correcting apparatus may comprise a first component having a main body configured for positioning between a surface of the external ear and a helix of the external ear. The main body may be configured to extend along at least a portion or length of the helix. The correcting apparatus may further comprise a second component having a first segment configured to contact a portion of a helical rim of the external ear and a second segment configured to lend support to the first component and extend posteriorly over the external ear when in use. This second component may have an arcuate-like shape. In some embodiments, the second segment may be configured to contact a skin surface posterior to the external ear. The first and second components may be coupled to each other, or they may be formed as a single unitary device.

The first component may be configured to reshape a fused helix of the external ear, such as when the helical rim has folded, moved or otherwise shifted, over the ear such that the helix is partially or completely fused to the antihelix. In this embodiment, the first component may comprise a main body having sufficient rigidity to remain in position during a time period of use sufficient to correct a deformity in the external ear. The main body may be preferably configured to move the helix away from the antihelix to create a scapha.

The correcting apparatus may be secured in place with the first component against the helix of the ear in a variety of manners. In one embodiment, an adhesive panel may be applied to the second component to secure it to the patient's scalp behind the ear, with the first component being positioned against the helix of the ear. The correcting apparatus may be configured to remodel the helical rim, for example, after the fused helix has been moved away from the antihelix. In this embodiment, the first segment may comprise an arcuate portion sized and shaped to remodel the outer helical rim after it has been moved away from the ear by the first component. In particular, the first segment of second correcting component helps to form the outer helical rim through remodeling or reshaping over a period of time.

In certain embodiments, the second component may comprise a posterior segment that is sized and shaped to extend posterior to the ear to a location on the scalp. The posterior segment may be attached to the skin surface of the scalp in a variety of manners, such as with the adhesive panel previously mentioned.

In another aspect, a correcting apparatus for remodeling a portion of an external ear may comprises a correcting component having a main body shaped for insertion into, or in contact with, a conchal bowl of the ear. The correcting component may also have first and second projections extending anteriorly from the main body. The first projection may be configured for positioning near, or in contact with, at least a portion of a cymba of the ear and the second projection may be configured to extend towards a tragus and into a cavum of the external ear. The main body preferably may comprise a material sufficiently rigid to remain in position during a time period of use sufficient to correct a deformity in the external ear. In certain embodiments, the correcting component helps to mold the conchal bowl by flattening a vertical or horizontal conchal crus and an anteverted conchal bowl/or widening a tight conchal bowl during use.

The first and second projections define channels or slits therebetween and provide additional flexibility to the main body such that it can be deformed slightly to fit within the conchal bowl of an infant ear. The projections may also allow the component to deform slightly during movement of the infant's ear, thereby providing more comfort and less trauma during use. In addition, they may provide additional stability to correcting component to assist in maintaining the correcting component in place within the conchal bowl.

In certain embodiments, the correcting apparatus may comprise a third projection extending anteriorly from the main body. The third projection may be formed, for example, by creating a slit or channel through the second projection to create two separate projections having a slit or channel therebetween. This slit or channel may allow for the passage of sound waves therethrough, as discussed below.

In an exemplary embodiment, the correcting component may include an upper portion and a lower portion. The lower portion may be configured for contacting the skin of the ear and may comprise a first surface, a second opposing surface and a hole extending through the first and second surfaces for passing sound waves therethrough. Providing this through hole allows the infant to hear sounds unimpeded while wearing the correcting component.

In certain embodiments, the lower portion of the main body further includes an internal cavity coupled to the through hole. The cavity in the main body may be configured to form an acoustic chamber around the hole to enhance the passage of sound waves therethrough.

In some embodiments, the shape of the correcting component allows it to remain in place within the conchal bowl for a sufficient period of time to reshape the conchal bowl and correct the deformity. In other embodiments, the correcting apparatus may further comprise an attachment component for securing the correcting component against the conchal bowl. In certain embodiments, the attachment component may comprise an adhesive film, layer or tape, such as surgical tape, or the like. In other embodiments, the correcting component may be further secured against the conchal bowl by another correcting component and/or attachment component.

In certain embodiments, the upper portion may include a projection or wall. The correcting component may be held in place using an attachment component in the form of an adhesive tape placed over the correcting component and having fingers that can secure to or around the ear and/or scalp. This adhesive tape may include cutouts that match with openings of the correcting component, and may include one opening that enables the projection or wall of the upper portion to extend therethrough the tape. This lock and key shaped configuration between the opening of the attachment component and the wall of the upper portion of the correcting apparatus further helps couple the correcting component and attachment component together, to avoid inadvertent dislodgment of the correcting component from the ear during use.

The conchal bowl correcting component preferably comprises a flexible, soft and conformable material, such as medical grade silicone, polymer, plastic, polymeric blend, or similar soft and pliable material suitable for human use. Similar to other embodiments, the correcting component may also include an inner portion that is formed of a more rigid material that has sufficient stiffness to maintain its shape and provide the necessary structural support and physical pressure on the patient's ear to allow the ear to remodel itself.

In another aspect, a correcting apparatus for remodeling a portion of an external ear comprises a correcting component having a main body shaped for extending across a portion of the external ear. The main body comprises a first portion configured for contacting a helix of the internal ear and a second portion configured for contacting an anterior surface of the external ear. The apparatus further includes a first attachment component coupled to the first portion of the main body and configured to attach the correcting component to a location posterior to the external ear and a second attachment component coupled to the second portion of the main body and configured to attach the correcting component to a location anterior to the external ear. In certain embodiments, the main body is configured to flatten out the external ear to correct for ear prominence or protruding ears.

In other embodiments, the correcting apparatus in this embodiment may be used in combination with other correcting components. For example, in one such embodiment, the correcting apparatus may be used in combination with a conchal bowl former, such as the one described above. In this embodiment, the main body may be configured to extend over at least a portion of the conchal bowl former to secure this component in position.

The first and second attachment components may be integral with the main body, or they may be coupled to the main body with a suitable adhesive. In certain embodiments, the first attachment component comprises a plurality of elongate members configured to extend from the first portion of the main body to a location on a scalp posterior to the external ear. The second attachment component may comprise one or more members configured to extend from the second portion of the main body to a location on a scalp anterior to the external ear or to exterior surfaces of the helix.

The attachment components may comprise any suitable adhesive device, such as surgical tape, Velcro or hook and loop materials, temporary glue and other such known skin attachment mechanisms. In one embodiment, the attachment components comprise a one-sided or double-sided surgical tape that is configured for adhering them to the outer skin. The first attachment member may, for example, be adhered to a portion of the face anterior to the external ear while the second attachment member is adhered to a portion of the scalp posterior to the external ear or to exterior surfaces of the helix.

In certain embodiments, the correcting component further comprises an elongate member extending superiorly from the first portion of the main body and configured to contact the helix. The elongate member may provide additional support for the correcting component. In addition, or alternatively, the elongate member may be configured to correct a deformity in the helix or helical rim.

The correcting component may further comprise a second elongate member extending inferiorly from the first portion of the main body and configured to contact an ear lobe. The second elongate member may provide additional support for the correcting component. In addition, or alternatively, the second elongate member may be configured to correct a deformity in the ear lobe.

In certain embodiments, the main body includes a first surface, a second opposing surface and a hole extending through the first and second surfaces for passing sound waves therethrough. Providing this through hole allows the infant to hear sounds unimpeded, while wearing the correcting component.

In an exemplary embodiment, the correcting component and the first and second attachment components comprise a first layer of soft deformable material and a second layer coupled to the first layer for maintaining a shape of the first layer. The first layer may comprise any suitable soft, atraumatic material for placement against the skin of an infant, such as silicone, polymer, plastic or a blend thereof. The first layer may also include an applied adhesive on the side facing the second layer. The second layer may comprise any suitable bendable, but substantially rigid, material to remain in position during a time period of use sufficient to correct the deformity, such as a metal, wire mesh or other bendable structure such as a sheet metal strip that can hold position, and the like.

The correcting component may include a third layer coupled to the second layer and configured to adhere the first and second layers together. In certain embodiments, the third layer comprises an adhesive tape. The adhesive tape may be a one-sided adhesive tape for securing the first and second layers together, or it may be a two-sided tape for additionally securing the component to the patient's outer skin. Alternatively, the component may comprise a fourth layer of adhesive tape that secures the component to the patient.

In another aspect, a correcting apparatus for remodeling a portion of an external ear comprises a correcting component configured for positioning within, or in contact with, a fossa triangularis of the external ear for correcting a deformity of the helix, the helical rim and/or the fossa (e.g., cryptotia or a missing or deformed triangular fossa). The correcting component includes a main body having an arcuate surface configured for contacting an inner surface of a helix and first and second support members extending from the main body.

In certain embodiments, the first and second support members are spaced from each other and define a channel or slit therebetween. This channel provides a certain degree of flexibility to the component during placement to minimize trauma to the patient. In addition, the support members provide stability to the main body to help hold it in position within the fossa.

The correcting apparatus is preferably shaped and configured to remain in place within the fossa for a sufficient period of time to correct the deformity. Alternatively, the correcting apparatus may further include an attachment component coupled to the correcting component for securing the component to the fossa. In one such embodiment, the attachment component comprises an adhesive element for adhering to a skin surface and a flexible element coupling the adhesive element to the correcting component. The flexible element is configured to extend from the correcting component over the helical rim of the external ear such that the adhesive element contacts a skin surface of the scalp posterior to the external ear.

In yet another aspect, a correcting apparatus for correcting a prominent ear lobe may be provided. The correcting apparatus may comprise a main body having a generally barbell, or in another embodiment, a generally bowling pin shape. One end of the main body may be larger than the other end. The correcting apparatus may be configured for attachment to a patient's ear lobe over a period of time of wear to create a flatter, better shaped ear lobe.

In another aspect, a correction system for remodeling a portion of an external ear comprises a first correcting component having a main body shaped for insertion into, or in contact with, a conchal bowl of the ear and a second correcting component having a main body configured for positioning between a surface of the external ear and a helix of the external ear.

In some embodiments, the correction system may further comprise a third correcting component configured for positioning within, or in contact with, a fossa triangularis of the external ear.

In some embodiments, the correction system may further comprise a fourth correcting component having a main body shaped for extending across a portion of the external ear, the main body comprises a first portion configured for contacting a helix of the internal ear and a second portion configured for contacting an anterior surface of the external ear.

In still another aspect, a kit for treating a deformity of an external ear is provided. This kit may include a correcting apparatus and/or correction system as previously described. In addition, the kit may include a guide for determining which of the correcting apparatuses or correction system to select for treating the deformity. This guide may comprise a transparency having an outline of a model human ear thereon. The outline of the model human ear may contain sections corresponding to at least one of the correcting apparatuses or correction system. The sections may be designated by lines, and/or also may be color coded.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1A illustrates a front view of an exemplary human right ear;

FIG. 1B illustrates a rear view of the ear of FIG. 1A;

FIG. 2A illustrates a partial cutaway side view of an exemplary embodiment of a correcting component for a helical rim of a human ear;

FIG. 2B illustrates a partial cutaway side view of another exemplary embodiment of a correcting component for a helical rim of a human ear;

FIG. 2C illustrates a partial cutaway side view of still another exemplary embodiment of a correcting component for a helical rim of a human ear;

FIG. 2D illustrates a partial cutaway side view of yet another exemplary embodiment of a correcting component for a helical rim of a human ear;

FIG. 3 illustrates a top-down view of an exemplary embodiment of an attachment component for use with the correcting components of FIGS. 2A to 2D;

FIG. 4 illustrates the correcting component of FIG. 2D in use with the attachment component of FIG. 3;

FIG. 5 illustrates a front perspective view of an exemplary embodiment of a correcting component for an antihelix;

FIG. 6 illustrates the correcting component of FIG. 5 in use behind an external ear of an infant;

FIG. 7A illustrates a perspective front view of an exemplary embodiment of a correcting component for a helical rim;

FIG. 7B illustrates a perspective bottom-up view of the correcting component of FIG. 7A;

FIG. 8A illustrates a perspective front view of an exemplary embodiment of an attachment component for use with the correcting component of FIG. 7A;

FIG. 8B illustrates the attachment component of FIG. 8A coupled to the correcting component of FIG. 7A;

FIG. 9 illustrates the correcting component and attachment component of FIG. 8B in use on an external ear of an infant;

FIG. 10A is a front perspective view of an exemplary embodiment of a correcting component for a conchal bowl of a right human ear;

FIG. 10B is a top-down view of the correcting component of FIG. 10A;

FIG. 10C is a bottom-up perspective view of the correcting component of FIG. 10A;

FIG. 10D is a top-down view of the correcting component of FIG. 10A configured for a left human ear;

FIG. 11 illustrates the correcting component of FIG. 10D in use and positioned within an external left ear of an infant;

FIG. 12 illustrates a top-down view of an exemplary embodiment of an attachment component for use with the correcting component of FIGS. 10A to 10D, configured for use with the correcting component of FIG. 10D for a left human ear;

FIG. 13 illustrates the attachment component of FIG. 13 in use with the correcting component of FIGS. 10A to 10C and positioned relative to an external right ear of an infant;

FIG. 14A illustrates a top-down view of another exemplary embodiment of an attachment component for use with the correcting component of FIG. 10D for infants with sensitive skin;

FIG. 14B is an exploded view of the attachment component of FIG. 14A;

FIG. 15 illustrates the correcting component of FIG. 10D in use with the attachment component of 14A in position relative to a left human ear of an infant;

FIG. 16 is a perspective view of an exemplary embodiment of a correcting component for correcting an ear lobe defect of a human ear;

FIG. 17A illustrates the correcting component of FIG. 16 in use with an exemplary embodiment of an attachment component;

FIG. 17B illustrates the correcting component of FIG. 16 in use with another exemplary embodiment of an attachment component;

FIG. 17C illustrates the correcting component of FIG. 16 in use with the attachment component of FIG. 13;

FIG. 18A illustrates a bottom-up perspective view of an exemplary embodiment of a correcting component for a triangular fossa of a human ear;

FIG. 18B illustrate a top-down perspective view of the correcting component of FIG. 18A;

FIG. 19 illustrates the correcting component of FIGS. 18A and 18B in use and positioned relative to an external left ear of an infant;

FIG. 20 illustrates the correcting component of FIGS. 18A and 18B in use with another exemplary embodiment of an attachment component;

FIG. 21 illustrates the correcting component and attachment component of FIG. 20 in use and attached to an external left ear of an infant;

FIG. 22A illustrates a top-down view of an exemplary embodiment of a correcting component for an ear lobe;

FIG. 22B illustrates a side perspective view of the correcting component of FIG. 22A; and

FIG. 23 illustrates the correcting component of FIGS. 22A and 22B attached to an external ear of an infant.

### DETAILED DESCRIPTION

Systems for remodeling and/or reshaping portions of the external ear and methods for their use are provided. These systems provide one or more non-surgical correcting apparatuses or components that may be worn externally to correct a deformity or malformation on the external ear through remodeling over a period of time. Using a typical infant's ear as a guide, each of the correcting components are configured to remodel and/or and reshape portions of the deformed ear and correct its present deformities. The systems offer a modular approach to the correction of the ear. Depending on the deformity or deformities being corrected, the correcting components may be used alone or in combination with other components of the systems to provide a comprehensive approach and an overall reshaping or remodeling of the ear. Methods of using these systems are described hereinbelow.

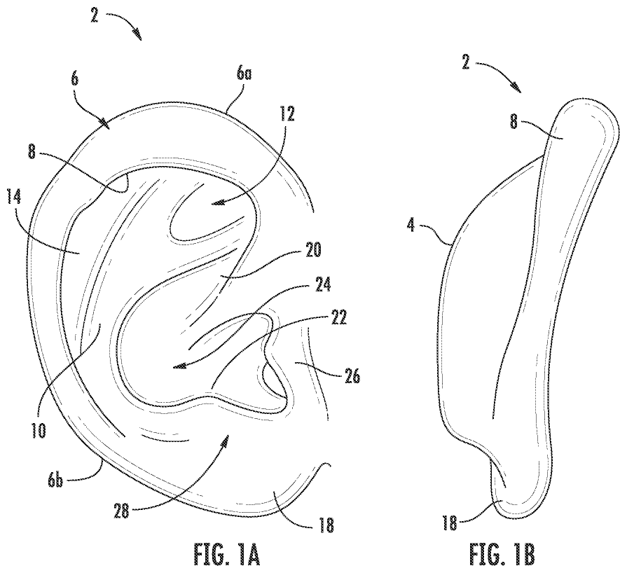

By way of background and for contextual perspective, FIGS. 1A and 1B illustrate a normal human ear. The figures may be referred to for anatomical context and perspective to appreciate and understand the correcting apparatuses of the present disclosure. In general, the visible part of the external ear is called the auricle. The auricle is also referred to as the pinna. The auricle is composed of a thin plate of cartilage. The cartilage is covered with skin. The cartilage is also connected to the surrounding parts by ligaments and muscles. Furthermore, the cartilage is connected to the commencement of the ear canal by fibrous tissue.

FIG. 1A shows a front view of a normal human right ear 2 and FIG. 1B shows the rear, or back, view of the same ear 2. The human ear 2 extends from a base 4 that attaches to the scalp skin (not shown). The major outer curved portion of the ear 2 is generally referred to as the helix 6. The helix 6 extends from a superior helix 6a to a descending helix 6b, as indicated in FIG. 1A. The ear 2 also includes a helical rim 8 and an antihelix 10 within the interior of the external ear 2 itself, as shown in FIG. 1A. The scapha 14 is the space between the helical rim 8 and the antihelix 10. The triangular fossa 12 is located at the upper inner portion of the ear 2. The ear 2 also includes the cymba 20 and cavum 22, which collectively can be generally referred to as the concha, or conchal bowl 24. Outside the conchal bowl 24 is the tragus 26 and the antitragus 28, while the bottom of the ear 2 extends into a lobule, or lobe 18.

As previously mentioned, infants are sometimes born with external ear deformities or malformations. External ear shaping mechanisms are known to exist. However, these existing ear shaping mechanisms do not always adequately address the unique concerns of newborns or young infants. For example, an ideal external ear remodeling mechanism would take into account the delicate skin of newborns, and avoid causing any harm or further deformities. Since the newborn and young infant ear is much smaller in scale than a normal human adult ear, the ear shaping mechanism needs to be appropriately sized and shaped as well as scaled (i.e., not too bulky or heavy) to be an effective treatment for a newborn or young infant.

The present disclosure provides various embodiments of correcting apparatuses that are configured to conform a shape of the deformed external ear of a newborn or infant to a shape of a model external ear when worn over a time period. In some embodiments, the correcting apparatus may be used in combination with an additional external ear component. The correcting apparatuses, along with any external ear components, are all considered modular components of correction systems to treat the external ear deformity. For example, helical rim correcting apparatuses, conchal bowl correcting apparatuses, structural arch correcting apparatuses, and fossa correcting apparatuses are described herein as components of modular correction systems, along with any associated external ear component for maintaining the apparatuses in place. In use, the correcting apparatuses or correction systems act as a remodeling guide, enveloping and applying compression or exerting pressure on the ear deformity while also providing a physical support. It is to be understood that what is meant by the term “remodeling” throughout this disclosure is the correction of the shape of the ear deformity.

These correcting apparatuses and correction systems may be made from a medical grade silicone, polymer, plastic, polymeric blend, or similar soft and pliable material suitable for human use. In some embodiments, the material may be transparent, translucent, or semi-opaque, and allow the physician to see the patient's ear through the apparatus or system during use to monitor progress. In some embodiments, the correcting apparatuses may also comprise an internal material to provide rigidity and/or bendability to the outer portion of the apparatus. The rigid material may be coated or embedded within a polymeric or plastic coating or layer to further ensure that a smooth and non-damaging surface is provided for the patient.

The remodeling and reshaping systems described herein provide an array of uniquely configured wearable components or devices for treating specific areas of the external ear. These wearable devices or correcting apparatuses may be configured for remodeling or reshaping a distinct region or anatomical location of the external ear, and may be used alone, or in combination with one another, as part of a comprehensive system to treat the ear. These components and apparatuses will now be described in detail below with respect to the specific area of the ear to be treated.

### Helix, Antihelix and Helical Rim

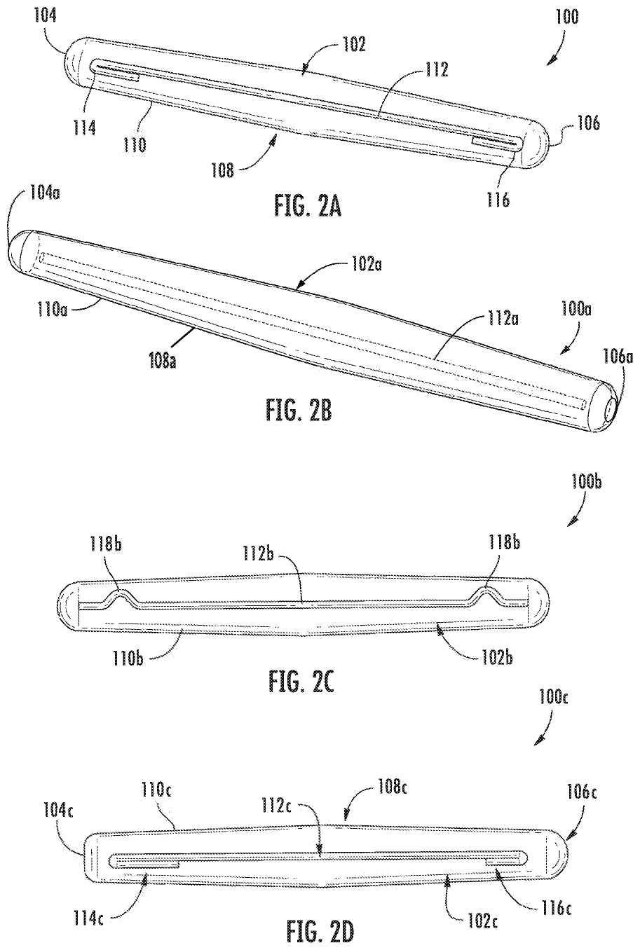

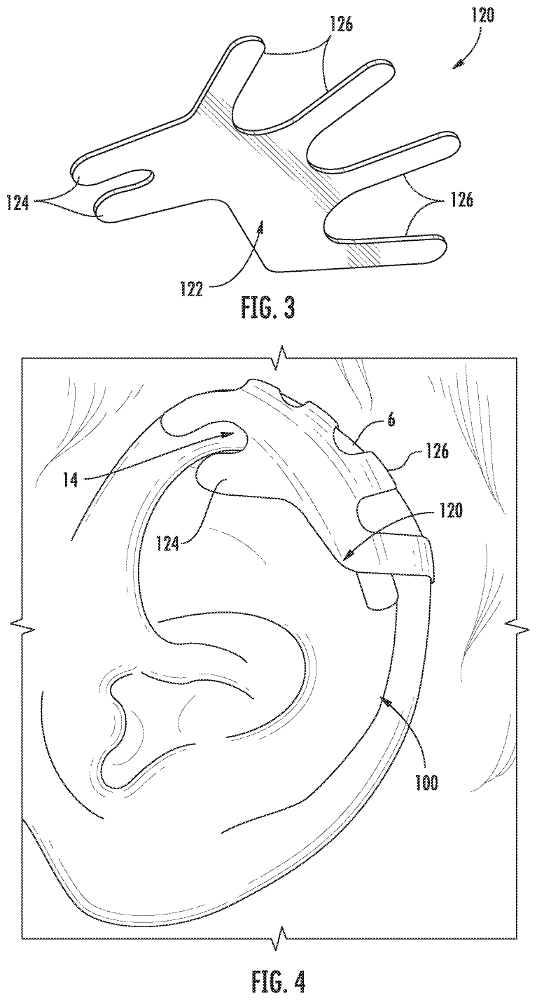

Turning now to the drawings, FIGS. 2A to 2D illustrate different embodiments of a correcting apparatus, or correcting component 100, for the reshaping and/or remodeling of the external ear. Correcting component 100 may include a main body 102 for placement within, or in contact with, a portion of the helix 6 of the ear 2. In certain embodiments, main body 102 may be placed at least partially in contact with the scapha 14 below or under the helical rim 8 in order to remodel or reshape the helix 6 and create or widen the scapha 14 as needed (see FIG. 4).

As shown in FIG. 2A, main body 102 preferably includes a generally elongate member having first and second ends 104, 106 that may have an atraumatic shape, e.g., rounded, dome-shaped or the like. Main body 102 may have a substantially cylindrical shape with a circular cross-section, or it may have other suitable cross-sections (e.g., oval, rectangular, square or the like). In certain embodiments, the central portion 108 of main body 102 may have a larger outer dimension (e.g., diameter, width or height depending on the cross-sectional shape) than the outer dimension of first and second ends 104, 106. This wider central portion 108 enables the main body 102 to preferentially create the scapha at its appropriate part of the helix—antihelix portion of the ear 2. Ends 104, 106 may have substantially the same outer dimension, first end 104 may have a larger outer dimension than second end 106 or vice versa. In use, the correcting apparatus 100 may be placed within the deformed helix 6 of the newborn or young infant for a time period sufficient to allow the deformed ear to remodel or become reshaped to an ideal target shape, and retain this shape after removal of the apparatus 100.

Main body 102 preferably comprises an outer portion 110 that comprises a flexible, soft and conformable material, such as medical grade silicone, polymer, plastic, polymeric blend, or similar soft and pliable material suitable for human use. Outer portion 110 ensures that a smooth and atraumatic surface is provided for contact with the patient's skin. Main body 102 further includes an inner portion 112 that is formed of a more rigid material that has sufficient stiffness to maintain its shape and provide the necessary structural support and physical pressure on the patient's ear to allow the ear to remodel itself. At the same time, inner portion 112 is flexible enough that it is capable of being molded or shaped during implementation. Inner portion 112 may comprise any suitable element that is sufficiently rigid and bendable for these purposes, such as a flexible wire, filament, wire mesh, or other bendable structure such as a sheet metal strip that can hold position, or the like, and may comprise any suitable material, such as stainless steel, tungsten, aluminum, polymers or the like.

In certain embodiments, as shown in FIG. 2A, inner portion 112 may be bent backwards or folded over at its two ends 114, 116 to reduce rotation of outer portion 110 around inner portion 112 during shaping or forming of correcting component 100. In addition, bending inner portion 112 backwards inhibits ends 114, 116 from piercing through main body 102, which could potentially injure the patient.

In one exemplary embodiment, as illustrated in FIG. 2B, the correcting apparatus 100a has all of the same features as correcting apparatus 100, including a main body 102a, first end 104a, second end 106a, and a central portion 108a, except that the inner portion 112a is a continuous metal wire or filament that extends along substantially the entire length of main body 102a from first end 104a to second end 106a.

In another exemplary embodiment, as illustrated in FIG. 2C, the correcting apparatus 100b has all of the same features as correcting apparatus 100, including a main body 102b with an inner portion 112b that extends along substantially the entire length of the main body 102b. However, inner portion 112b includes one or more bends 118b near each end, as illustrated, to reduce rotation of outer portion 110b and inhibit inner portion 112b from piercing through outer portion 110b, which could potentially injure the patient.

Referring now to FIG. 2D, another embodiment of a correcting component 100c preferably comprises a generally elongate member having first and second ends 104c, 106c. In this embodiment, second end 106c has a more rounded shape than first end 104c. In addition, second end 106c tapers further away from main body 102c than first end 104c. Second end 106c is designed to fit along the descending portion of the helix, and the tapered shape allows end 106c to fit in this area more precisely. First end 104c is designed to fit within the superior helix of the ear.

As in the embodiment shown in FIG. 2A, inner portion 112c bends backwards or is folded over at its two ends 114c, 116c to reduce rotation of outer portion 110c and inhibit inner portion 112c from piercing through outer portion 110c. This may also provide additional rigidity to the ends 104c, 106c of main body 102c and allow correcting component 100c to be deformed to fit the patient's anatomy (e.g., by bending central portion 108c such that main body 102c has an arcuate shape that may conform to a helix of a patient). In this embodiment, however, inner portion 112c is asymmetric, such that end 114c bends backward with a longer length than end 116c to facilitate localized bending of component 100c.

For all of the correcting components 100, 100a, 100b, 100c of FIGS. 2A to 2D, the inner portion 112 provides additional rigidity to ends 104, 106 of main body 102 to allow correcting component 100 to be deformed to fit the patient's anatomy (e.g., by bending central portion 108 such that main body 102 has an arcuate shape that may conform to a helix of a patient). The main body 102 preferably comprises an outer portion 110 that comprises a flexible, soft and conformable material, such as medical grade silicone, polymer, plastic, polymeric blend, or similar soft and pliable material suitable for human use. The main body 102 further includes an inner portion 112 that is formed of a more rigid material that has sufficient stiffness to maintain its shape and provide the necessary structural support and physical pressure on the patient's ear to allow the ear to remodel itself.

The main body 102 may be provided with a preformed shape corresponding to a desired shape of a normal human ear. Alternatively, the main body 102 may be molded or shaped during implementation. For example, the inner portion 112 of main body 102 may be bendable and moldable, and able to maintain its shape after molding, such that the physician can sculpt the main body 102 of the correcting component 100 into the desired ear shape on the patient's ear. This design also allows for incremental shaping since the physician would have the ability to shape the main body 102 incrementally over a time period to incrementally match the patient's ear to the shape of the model external ear. This feature would be especially helpful in cases of severe deformity or where the deformities may exist in distinct separate locations on the patient's ear.

It is contemplated that, although inner portion 112, 112a, 112b, 112c is shown as a substantially continuous, single component in FIGS. 2A to 2D, the invention is not limited to this configuration. For example, inner portion 112, 112a, 112b, 112c may comprise a plurality of separate components embedded at different locations within correcting component 100, 100a, 100b, 100c.

Referring now to FIG. 3, an attachment component 120 for use with the correcting component 100, 100a, 100b, 100c of FIGS. 2A to 2D will now be described. In an exemplary embodiment, the attachment component 120 comprises an adhesive, such as a surgical tape, for directly coupling to the patient's skin. Of course, while a surgical tape is described and shown, it is understood that other adhesives may also be employed, such as for example, temporary glues, surgical tapes, Velcro or hook and loop materials, and other such known skin attachment mechanisms. The attachment component should ideally be strong enough to temporarily align correcting component 100 with attachment component 120 during installation of the device (as will be discussed in more detail below), but not so strong that damage to the skin occurs when the attachment component 120 is removed.

As shown, attachment component 120 comprises a main body 122 with a generally arcuate shape that is intended to correspond with the shape of main body 102 of correcting component 100 when it has been deformed to the shape of helix 6 of the patient's ear (see FIG. 4). Attachment component 120 further comprises one or more inner flanges or projections 124 and one more outer fingers or projections 126. Inner projections 124 are designed to extend anteriorly from main body 102 when attachment component is in place on the external ear (see FIG. 4).

Outer projections 126 are shaped and designed to wrap around the helix 6 of the ear 2 to hold attachment component 120 and correcting component 100 in place (see FIG. 4). As such, the attachment component 120 may be seen as an overlay or protective covering in addition to an attachment mechanism for the correcting component. In one exemplary embodiment, outer projections 126 comprises elongate fingers that extend substantially posteriorly from main body 122. Outer projections 126 are preferably spaced from each other and aligned with main body 102 such that they provide sufficient support to maintain correcting component 100 within helix 6 of the ear. Outer projections 126 have a length selected such that the flanges 126 may wrap around helix 6 to the back of the ear. Although four outer flanges are shown in the drawings, it should be recognized that attachment component 120 may comprise any suitable number of outer flanges in order to secure correcting component 100 in place (i.e., preferably around 2 to 10 flanges, more preferably between about 3 to 6).

In certain embodiments, inner projections 124 are designed to extend to a surface of the external ear, such as the antihelix 10, cymba 20, cavum 22, tragus 26 or other suitable surface, to hold attachment component 120 and correcting component 100 in place. Further, the attachment component 120 may contain inner projections 124 with entirely different shapes and configurations, such as square, rounded, oval, rectangular and the like.

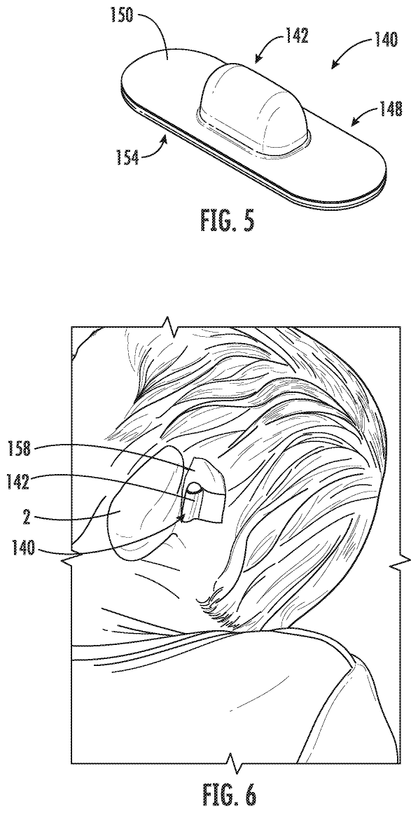

FIG. 5 illustrates an exemplary embodiment of an antihelix correcting component 140. The antihelix correcting component 140 may comprise an anti-helical dome 142 atop a base or panel 148. The dome 142 may be elongate, as shown, as well as the panel 148. It is contemplated that the dome 142 and panel 148 may be provided as one unitary component, or as two separate pieces attached together. The dome 142 may be formed of a material such as a plastic or polymer, and may be a flexible, soft and conformable material, such as medical grade silicone, polymer, plastic, polymeric blend, or similar soft and pliable material suitable for human use.

The base or panel 148 may be provided with an attachment mechanism on the surface opposite 154 to the surface 150 on which the anti-helical dome 142 sits. This attachment mechanism may be similar to the ones described herein for other attachment components, and can include adhesive films, layers or tapes, such as surgical tape, glue, Velcro, and the like. To use the antihelix correcting component 140, the antihelix correcting component 140 can be placed behind the ear, as shown in FIG. 6, using a tape 158 to keep the component in place. Over a period of time, the anti-helical dome 142 will press against the ear and create the antihelix anatomical structure with extended wear.

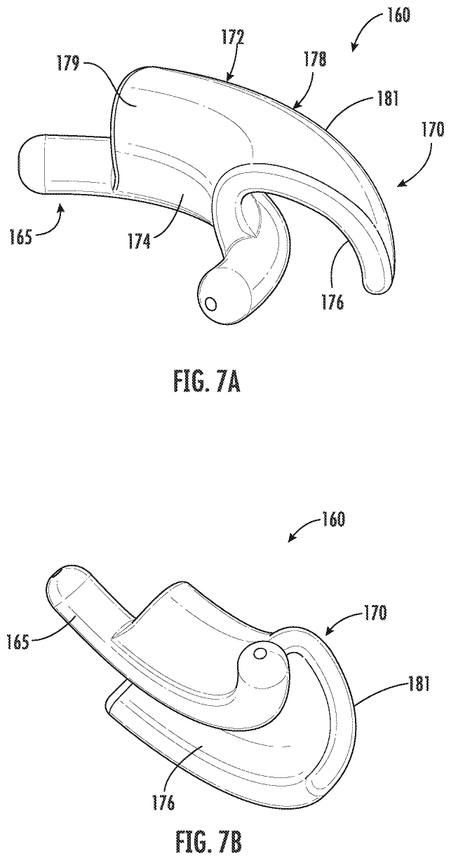

Referring to FIGS. 7A and 7B, another embodiment of a correcting apparatus 160 will now be described. Correcting apparatus 160 comprises a first helix correcting component 165 and a second helix correcting component 170. First helix correcting component 165 may be used to correct a partially or completely fused helix, wherein the helical rim has folded, moved or otherwise shifted, over the ear such that the helix is partially or completely fused against a surface of the ear, such as the scapha. Correcting component 165 is configured to open up the scapha to help form the helical rim. Second correcting component 170 is configured to correct a deformity of the helix or helical rim by, for example, forming the outer rim after correcting component 165 has opened it up and moved it away from the surface of the ear, e.g., the scapha. As such, the correcting apparatus 160 may be viewed as a whole as a constriction sheath in its function and operation on the ear.

Correcting apparatus 160 is preferably formed of a more rigid material than outer portion 110 of component 100. This material has sufficient stiffness to maintain its shape and provide the necessary structural support and physical pressure on the patient's ear to move the helix away from the surface of the external ear, e.g., the scapha. In certain embodiments, correcting apparatus 160 is sufficiently rigid that it will be pre-formed into the appropriate size and shape to perform its function on an individual ear. In other embodiments, correcting apparatus 160 is flexible enough that it is capable of being molded or shaped during implementation.

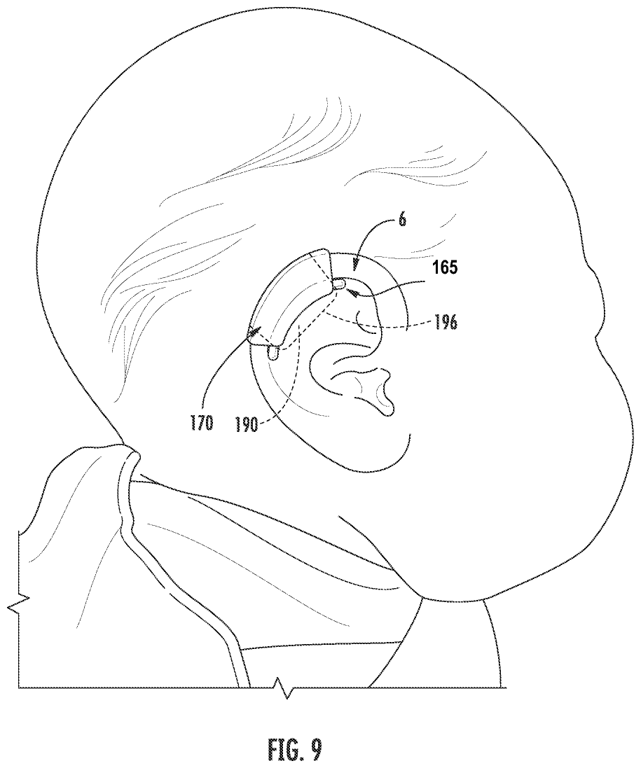

Second correcting component 170 comprises a main body 172 with a first end 174, a second end 176 and a substantially arcuate portion 178 therebetween. First end 174 is coupled to a central portion of first helix correcting component 165. In one embodiment, correcting components 165 and 170 may be molded together in a single unitary component. Arcuate portion 178 is sized and shaped to extend around a helical rim 6 of an external ear such that second end 176 contacts a surface of the scalp posterior to the ear (see FIG. 9). In addition, the inner surface of correcting component 170 serves to mold an outer portion of the helical rim.

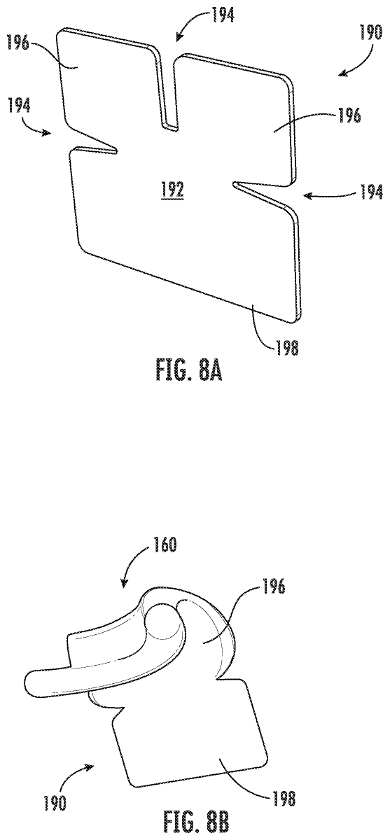

Arcuate portion 178 of correcting component 170 includes anterior and posterior segments 179, 181. Anterior segment 179 has been sized and shaped to remodel the helical rim after it has been moved away from the ear by correcting component 165. In particular, anterior segment 179 helps to form the outer helical rim through remodeling or reshaping over a period of time. Posterior segment 181 has been sized and shaped such that the second end 176 extends posterior to the ear toward the scalp. Correcting component 170 may be attached to the skin surface of the scalp in a variety of manners. In one embodiment, a double-sided tape 190 (see FIGS. 8A and 8B) is adhered to second end 176 which is then adhered to the scalp (see FIG. 9). Alternatively, component 170 may include an adhesive substance on second end 176 to directly adhere component 170 to the skin surface.

Correcting apparatus 160 may be secured within the helix of the ear in a variety of manners. In one embodiment, an adhesive panel or double-sided tape 190 as shown in FIG. 8A is applied to correcting apparatus 160 to secure it to the target location against the ear. The adhesive panel 190 may comprise a main body 192 having cutout portions 194 to create flaps 196, 198. As shown in FIG. 8B, the flaps 196 may be secured on the inside of the second helix correcting component at the posterior segment 181. Flap 198 may be used to secure the correcting apparatus 160 to the skin surface behind the ear, as shown in FIG. 9.

It should be noted that while correcting components 165, 170 have been shown together, they may be used as separate components. For example, correcting component 165 may be secured to the patient's ear to correct a fused helix by itself, e.g., through glue, surgical tape or the like. Similarly, correcting component 170 may be secured to the patient's ear, with glue, surgical tape or the like, to remodel or otherwise shape the helical rim. Alternatively, both components may be used together without coupling the components to each other.

### Conchal Bowl

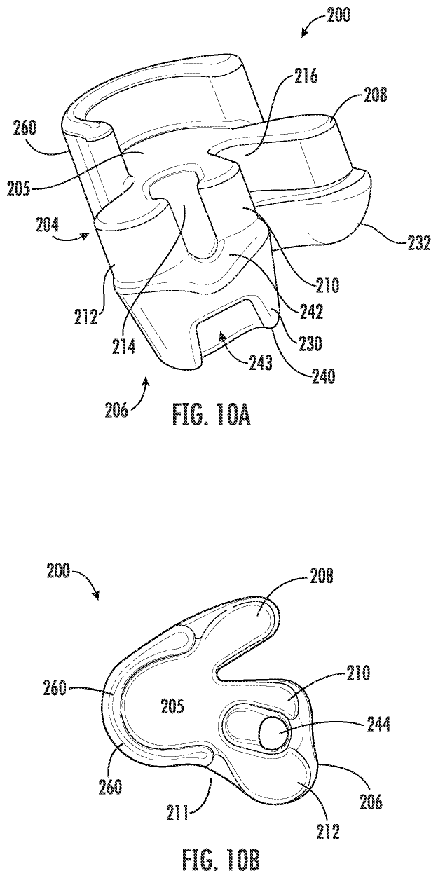

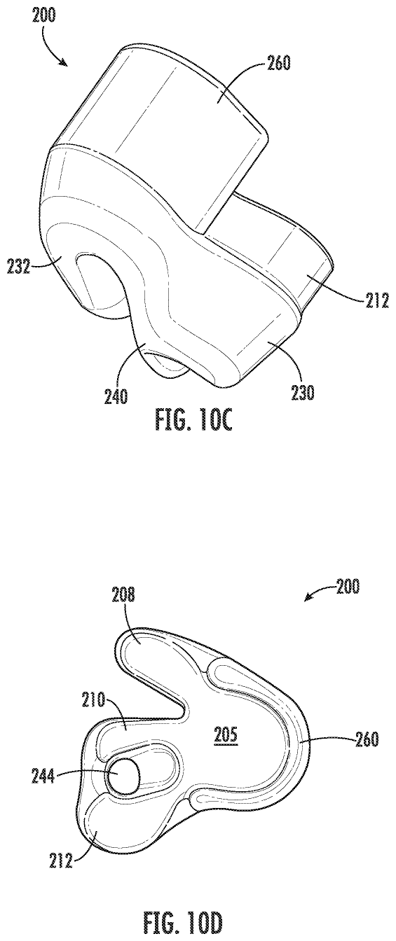

Referring to FIGS. 10A to 10D, a correcting apparatus or component 200 configured as a conchal bowl insert will now be described. Correcting component 200 comprises an upper portion 204 and a lower portion 206. Upper portion 204 includes a main body 205 and first, second and third projections 208, 210, 212 extending therefrom. Second and third projections 210, 212 are preferably initially shaped as one-piece or a single projection that has a slit or opening formed therein to allow for the passage of sound waves therethrough (discussed below). In certain embodiments, main body 205 may only include two such projections: a first projection 208 and a second projection (not shown) that combines the second and third projections 210, 212 shown in FIGS. 10A to 10D. In this embodiment, there is no slit or opening formed in the second projection.

The main body 205 is generally dimensioned to fit near or against the back wall of the conchal bowl 24 of the ear. First projection 208 extends anteriorly from main body 205 and has an elongate, slightly curved, shape generally configured to fit near or against the cymba 20 (or between the cymba 20 and the antihelix 10). Second and third projections 210, 212 extend anteriorly from main body 205 towards the tragus 26 of the ear and into the cavum 22. Collectively, the cymba and cavum form the concha or conchal bowl 24. Correcting component 200 helps to mold the conchal bowl by flattening a vertical or horizontal conchal crus and often widening a tight conchal bowl during use. In addition, correcting component 200 may help mold the conchal bowl 24 into a more concave shape (e.g., when the conchal bowl is misshapen as convex).

Upper and/or lower portions 204, 206 of component 200 may include a curved surface 211 designed to remodel the antitragus 28. Specifically, curved surface 211 is sized and shaped to move the antitragus 28 back down (e.g., in the direction of ear lobe 18) into its appropriate anatomical position when, for example, the antitragus has overgrown or otherwise been deformed upwards towards the conchal bowl 24 or the tragus 26.

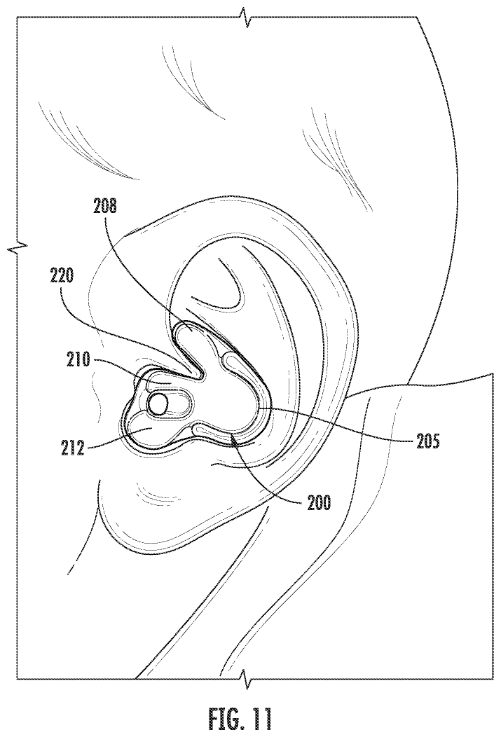

Projections 208, 210, 212 define open channels 214, 216 therebetween and serve to provide both flexibility and stability to component 200. The projections are deformable and bendable and function to provide additional flexibility to component 200 such that it can be deformed slightly to fit within the conchal bowl of an infant ear. The projections also allow component 200 to deform slightly during movement of the infant's ear, thereby providing more comfort and less trauma during use. In addition, they provide stability to component 200 because they extend on either side of the anterior portion 220 of the helical crus (see FIG. 11), which assists in maintaining correcting component 200 in place within the conchal bowl.

Lower portion 206 of correcting component 200 includes a main body 230 and a stem 232 extending therefrom. First projection 208 overlies stem 232 and second and third projections 210, 212 overlie main body 230. Upper and lower portions 204, 206 may be coupled to each other, or they may be molded together as a single unitary component.

Main body 230 of lower portion 206 includes a first surface 240 and a second opposing surface 242. In certain embodiments, main body 230 of lower portion 206 includes an opening or hole 243 that extends completely through main body 230 from first surface 240 to second surface 242. Opening 243 provides a passage through correcting component 200 for sound waves to pass therethrough, allowing the patient to hear sounds while component 200 is installed. Opening 243 is configured for positioning near, or over, the ear canal of the patient to facilitate passage of sounds waves through opening 243 and into the ear canal. In certain embodiments, opening 243 may be coupled to, or formed within, a larger cavity 244 in main body 230. In certain embodiments, cavity 244 may provide an acoustic chamber around opening 243 to enhance the passage of sound waves therethrough.

In certain embodiments, stem 232 further includes an internal cavity (not shown). The internal cavity may be a substantially elongate channel that extends through stem 232 and opens up into a chamber (not shown) having a generally circular cross-section. The internal cavity functions to provide additional flexibility to stem 232 such that it can be deformed slightly to fit within the conchal bowl of an infant ear. The cavity also allows stem 232 to deform slightly during movement of the infant's ear, thereby providing more comfort and less trauma during use.

As shown, an additional support element 260 may be coupled to, or integral with, upper and lower portions 204, 206 of the correcting component 200. Support element 260 provides a shaped extension tab extending from the upper portion 204 of the main body 205, which assists in placing the conchal bowl former correctly into the conchal bowl. It also provides additional support in molding the back wall of the conchal bowl when it is too shallow or is deformed. In addition, the support element 260 provides an extension over which an adhesive overlay may be placed, which will be described in more detail below.

FIGS. 10A, 10B and 10C illustrate a conchal bowl former correcting component 200 for the right human ear, while FIG. 10D illustrates a conchal bowl former correcting component 200 for the left human ear. It is understood that the component 200 of FIG. 10D is identical to the one for FIGS. 10A to 10C, except that it is a mirror image.

Correcting component 200 preferably comprises a flexible, soft and conformable material, such as medical grade silicone, polymer, plastic, polymeric blend, or similar soft and pliable material suitable for human use. Similar to other embodiments, correcting component may further include an inner portion (not shown) that is formed of a more rigid material that has sufficient stiffness to maintain its shape and provide the necessary structural support and physical pressure on the patient's ear to allow the ear to remodel.

In some embodiments, the shape of second correcting component 200 allows it to remain in place within the conchal bowl 24 for a sufficient period of time to reshape the conchal bowl 24 and correct the deformity. In other embodiments, one or more attachment components may be used to anchor correcting component 200 in place. In one such embodiment, an adhesive (not shown), such as glue, surgical tape, or the like, is applied to component to secure it within the conchal bowl. In other embodiments, an additional correcting component and/or attachment component may be used.

Correcting component 200 may be used in combination with other correcting components of the present systems. For example, the conchal bowl correcting component 200 may be used in combination with a helical rim correcting component 100 (as shown in FIG. 13), an antihelix correcting component 140, or a helix correcting component 160 as previously described.

While the present modular correction systems provide the ability to select one or more of the various correcting apparatuses to be applied to the patient's ear, Applicant has discovered that, in most cases, remodeling the conchal bowl with correcting component 200 facilitates the remodeling of other portions of the external ear. The conchal bowl appears to be an important driver towards the overall shape of these other portions of the ear. As the most dominant portion of the human ear, in terms of size, the conchal bowl may be viewed as the anchor to which the other parts of the ear attach. It can reasonably be understood, then, that any overall reshaping and/or remodeling of the ear would need to rely on a normally shaped conchal bowl in order to correct the shape of other attached parts of the ear.

### Structural Arch

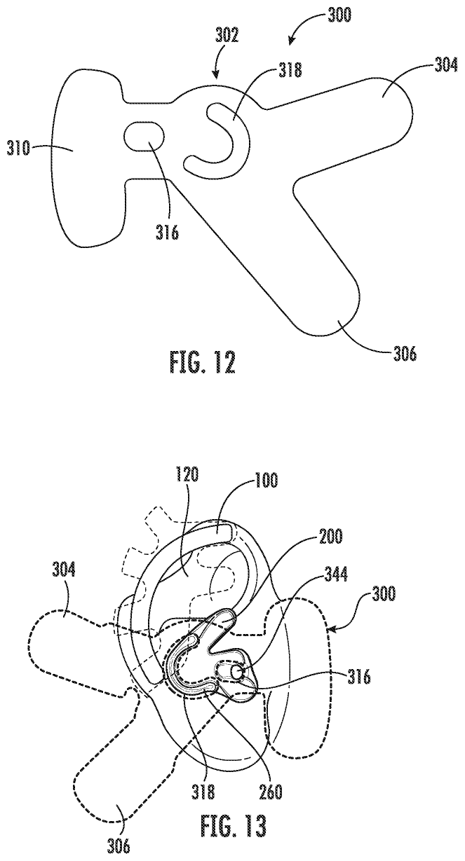

Referring now to FIGS. 12 and 13, an exemplary embodiment of an attachment component 300 for use with the conchal bowl correcting component 200 is illustrated. This attachment component serves a dual function to both maintain the conchal bowl correcting component 200 in place as a conchal bowl correcting component overlay, and also to serve as a structural arch correcting component for reshaping and/or remodeling portions of the external ear, as will now be described. Attachment component 300 includes a main body 302 configured to extend across a portion of the external ear and first and second elongate members 304, 306 generally configured to extend superiorly and inferiorly, respectively, from main body 302 along the helix 6 (see FIG. 13). Component 300 further includes an anterior attachment member 310 extending anteriorly from main body 302. In this embodiment, first, upper elongate member 304 and second, lower elongate member 306 may together form a generally “C” or “Y” shape (as shown). The upper elongate member 304 and lower elongate member 306 of a “Y” shaped embodiment may be the same length or they may be different in length.

Attachment component 300 may further include an opening or channel 316 extending therethrough, as shown in FIG. 12. Opening 316 is configured for positioning near, or over, the ear canal of the patient to facilitate passage of sounds waves through opening 316 and into the ear canal. The opening 316 may be positioned to align with the opening 244 of the conchal bowl correcting component 200. Additionally, the conchal bowl correcting component overlay/structural arch attachment component 300 may include a shaped slot 318 extending therethrough which allows the passage of the structural element or extending tab 260 of the conchal bowl correcting component 200, as illustrated in FIG. 13. The lock-and-key fit of the shaped slot 318 on the structural element 260 ensures that the component or overlay 300 remains properly aligned with the conchal bowl correcting component 200 at all times.

Attachment members 304, 306, 310 may comprise any suitable adhesive device, such as surgical tape, Velcro or hook and loop materials, temporary glue and other such known skin attachment mechanisms. In one embodiment, attachment members 304, 306, 310 comprise a one-sided or double-sided surgical tape that is configured for adhering them to the skin. Attachment member 310 may, for example, be adhered to a portion of the face anterior to the external ear while attachment members 304, 306 are adhered to a portion of the scalp posterior to the external ear (see FIG. 13). In an alternative embodiment (not shown), attachment members 304, 306 are wrapped around a portion of the helix and adhered to the back of the ear. The surgical tape may include an acrylic pressure sensitive adhesive and/or a release liner coated on one side with a silicone release coating.

It is contemplated that in certain situations, attachment component 300 may be used alone to flatten out the external ear to correct for one or more ear deformities, such as ear prominence. In certain cases, an underdeveloped anti helical fold may cause the helix to protrude outward. Ear prominence may also be caused by a deep concha which pushes the ear away from the side of the head. In addition, upper elongate member 304 may be shaped and positioned to flatten the upper part of the helix and/or to correct other helical rim deformities. Lower elongate member 306 may be shaped and positioned to flatten out the earlobe and/or correct for other earlobe deformities.

In addition (such as on the opposite ear), or in the alternative, attachment component 300 may be used in combination with other correcting components. As described and shown in FIG. 13, the attachment component 300 may be used in combination with a conchal bowl correcting component 200, such as the one described above. In this embodiment, main body 302 may be configured to extend over at least a portion of the conchal bowl correcting component 200 to secure this component in position. Additional correcting components may also be utilized without causing any interference, such as the helical rim correcting component 100 with its attachment component 120, as also illustrated in FIG. 13. This construct whereby multiple correcting components are used at the same time illustrates one of the key benefits of the modular remodeling and reshaping system provided herein. The ability to select from various correcting components targeting different treatment areas and utilize them at the same time offers a flexible and comprehensive treatment plan for the patient, allowing multiple treatments to be applied simultaneously to reduce the overall treatment time.

The simplified “C” or “Y” geometry of the attachment component 300 may be appropriate when the component is not wrapped over the rim of the ear, but instead secured against the patient's skin and across the ear in a generally flat or planar manner. In this approach, the attachment component 300 holds the ear against the patient's head, with the main body 302 positioned over the ear and the upper and lower elongate members 304, 306 extending past the ear rim to the patient's skin behind the ear. The attachment component 300 can therefore be used to press the ear against the skull for remodeling if so desired, and/or serve as a protective shield to further secure any components of the system to remain in its position, such as for example, the helical rim correcting component 100 and/or the conchal bowl correcting component 200. In this way, the attachment component 300 also functions as a structural arch correcting component as well as an attachment mechanism.

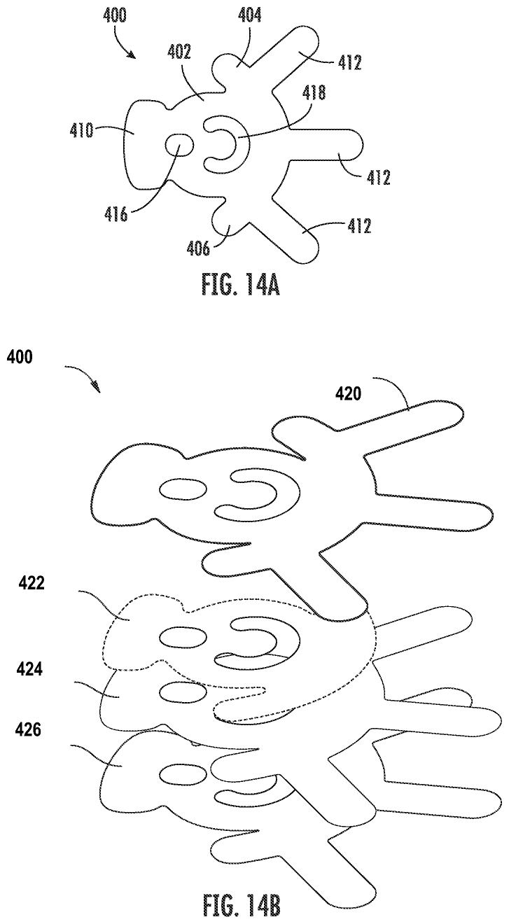

Referring now to FIGS. 14A and 14B, a similar attachment component 400 to attachment component 300 described above may be provided for protection of sensitive skin. This attachment component 400 (which also may serve as a structural arch correcting component and/or a conchal bowl correcting component 200 overlay in the same way attachment component 300 does) may include a main body 402 configured to extend across a portion of the external ear and first and second elongate members 404, 406. Additionally, one or more posterior attachment members or fingers 412 may extend posteriorly from the main body 402.

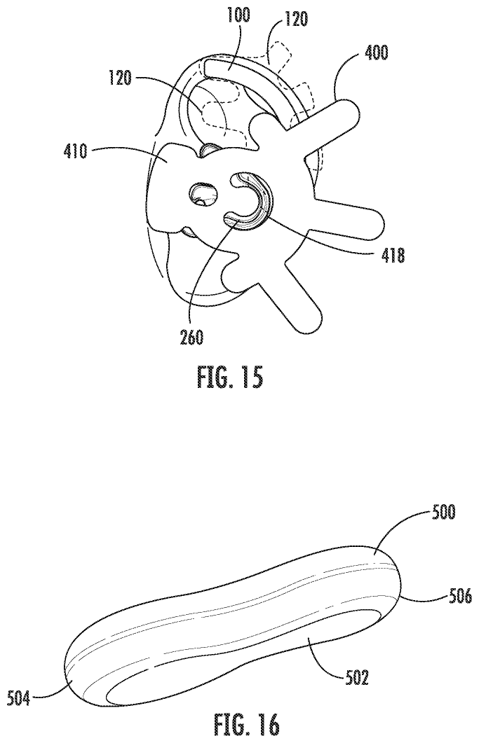

Attachment component 400 may further include an opening or channel 416 extending therethrough, as shown in FIG. 14A. Like attachment component 300, opening 416 is configured for positioning near, or over, the ear canal of the patient to facilitate passage of sounds waves through opening 416 and into the ear canal. The opening 416 may be positioned to align with the opening 244 of the conchal bowl correcting component 200. Additionally, the attachment component 400 may include a shaped slot 418 extending therethrough which allows the passage of the structural element or extending tab 260 of the conchal bowl correcting component 200, as illustrated in FIG. 15. The lock-and-key fit of the shaped slot 418 on the structural element 260 ensures that the component or overlay 400 remains properly aligned with the conchal bowl correcting component 200 at all times.

The attachment component 400 may comprise a plurality of layers that are bonded together to form the final component. In one embodiment, attachment component 400 includes at least a first outer layer 420, a second middle layer 422 and a third inner layer 424. Outer layer 420 preferably comprises a flexible, soft and conformable material, such as medical grade silicone, polymer, plastic, polymeric blend, or similar soft and pliable material suitable for human use. Middle layer 422 may be formed of a more rigid material, such as metal, certain polymers and the like, that has sufficient stiffness to maintain its shape and provide the necessary structural support and physical pressure on the patient's ear to hold attachment component 400 against the ear.

Inner layer 424 comprises an adhesive layer that includes an adhesive for bonding to middle and/or outer layers 422, 420, such as a pressure sensitive adhesive, surgical tape or the like. In certain embodiments, inner layer 424 may include an additional adhesive 426 on the attachment members 410, 412, such as a double-sided transparent polyethylene, for adhering to both the other layers of component 400 and to the patient's skin.

In an alternative embodiment, middle layer 422 may comprise an inserted mold with an outer layer of silicone or a similar material. The outer housing would replace inner layer 424 and outer layer 420. The outer housing services to trap middle layer 422 in place and may comprise an adhesive layer on the inside face(s) of the molded outer housing.

Sensitive skin attachment component 400 may be used in in a similar manner as attachment component 300 to secure the conchal bowl correcting component 200, as illustrated in FIG. 15. The sensitive skin attachment component 400 may be positioned over the correcting component(s), such as the helical rim correcting component 100 and its associated attachment component 120 (shown partially wrapped around the ear for ease of view only, though it is understood that the attachment component 120 is intended to wrap fully around the rim), and the conchal bowl correcting component 200. The extended tab 260 projecting from the conchal bowl correcting component 200 may extend through the shaped slot 418 of the attachment component 400, as shown.

### Ear Lobe

Referring now to FIG. 16, a correcting component 500 for an ear lobe will now be described. Correcting component 500 may be configured as an ear lobe boot to correct a variety of earlobe deformities, such as earlobe prominence, an abnormally shaped earlobe and the like. As shown, correcting component 500 includes a main body 502 generally shaped like a peanut or figure “8” or bowling pin, with one end 504 being larger than the other end 506. The main body 502 may be formed of a flexible, soft and conformable material, such as medical grade silicone, polymer, plastic, polymeric blend, or similar soft and pliable material suitable for human use. The correcting apparatus may be configured for attachment to a patient's ear lobe over a period of time of wear to create a flatter, better shaped ear lobe.

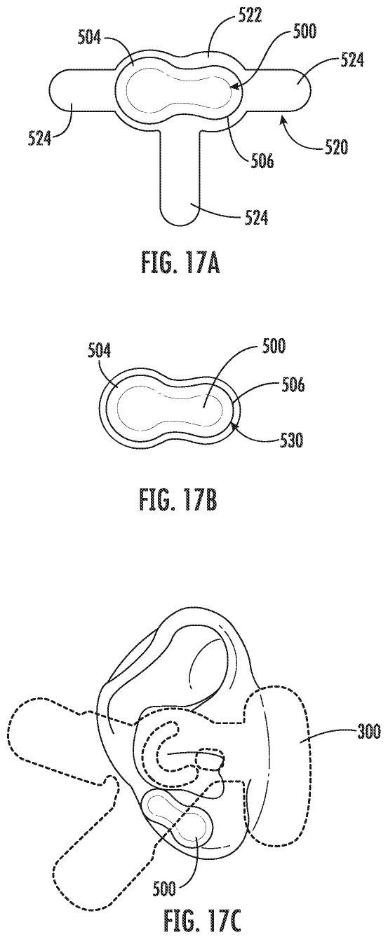

The correcting component 500 may be attached to the ear in a number of different ways. In one embodiment shown in FIG. 17A, an attachment component 520 may be provided. The attachment component 520 may be an adhesive tape similar to the ones previously described for attachment components 120, 158 and 190. The attachment component 520 may include a main body 522 upon which the ear lobe boot or correcting component 500 may be attached. One or more attachment members 524 may extend from the main body 522 for use in attaching the ear lobe boot or correcting component 500 to the ear 2.

In an alternative embodiment, the ear lobe boot or correcting component 500 may be secured to the ear 2 using a tape 530 that has a shape similar to the correcting component 500 itself, without any attachment members, as shown in FIG. 17B. As understood, the correcting component 500 may additionally be kept in place with any of the attachment components previously described, including attachment component 300, as shown in FIG. 17C. Of course, attachment component 400 may also be used for this purpose, either as a standalone, or in combination with attachment component 300, as described and illustrated in other examples.

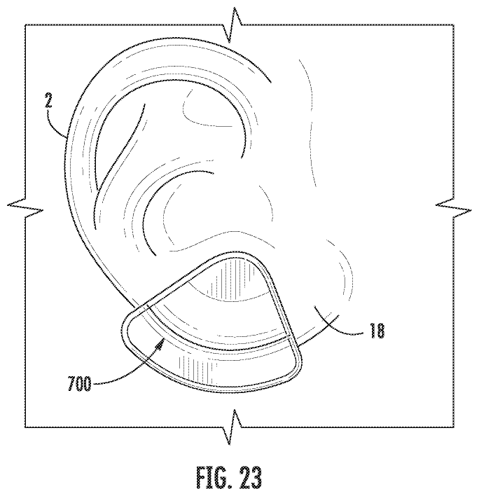

Another embodiment of an ear lobe correcting component 700 is illustrated in FIGS. 22A and 22B. The correcting component 700 may include a main body 702 and a flange 704 extending from main body 702. Main body 702 preferably comprises a lower surface 706 configured for contacting a surface of the outer skin below the ear lobe and an arcuate inner surface 708 configured to contacting a lower surface of the ear lobe of an external ear (see FIG. 23). Lower surface 706 may be adhered to the outer surface of the skin through any suitable adhesive, such as glue, tape or the like.

Flange 704 may extend upward and laterally from an upper surface 710 of main body 702 and configured to contact and engage an outer surface of the ear lobe when main body 702 is adhered to a suitable location on the skin surface. Flange 704 may preferably comprise a generally triangular shape with a rounded tip 712 and a curved outer surface 714 that generally corresponds to the arcuate shape of inner surface 708 of main body 702.

### Triangular Fossa

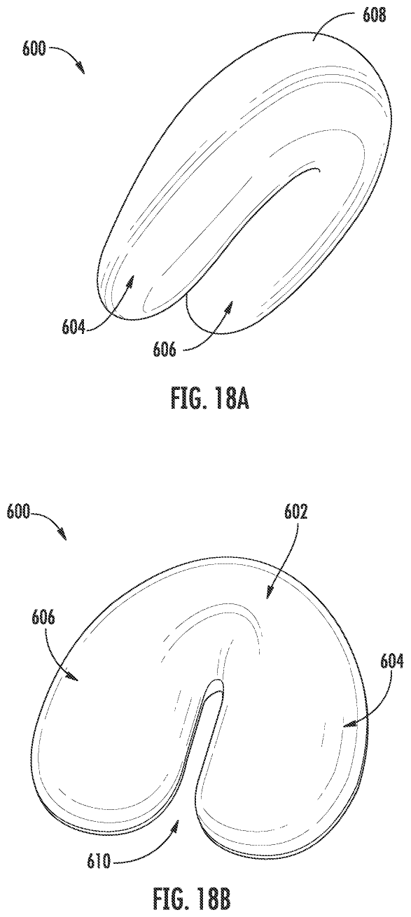

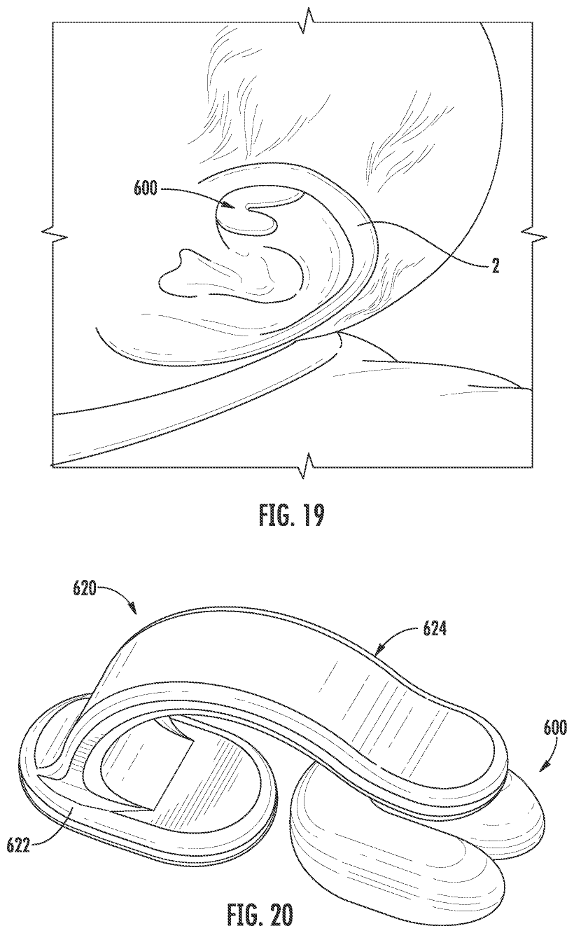

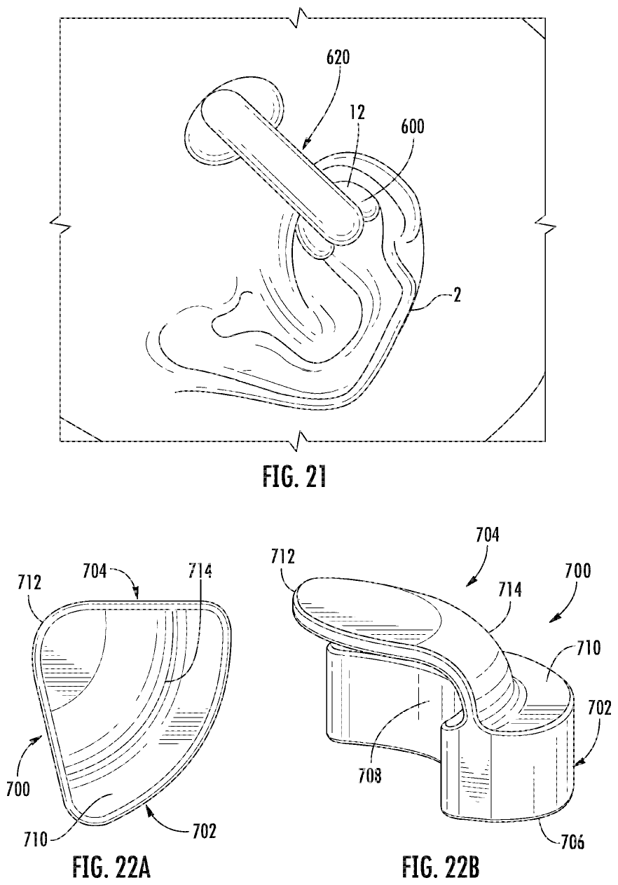

Referring to FIGS. 18A and 18B, a triangular fossa/cryptotia correcting apparatus or fossa correcting component 600 will now be described. Correcting component 600 preferably comprises a flexible, soft and conformable material, such as medical grade silicone, polymer, plastic, polymeric blend, or similar soft and pliable material suitable for human use. Correcting component 600 is designed to fit into the triangular fossa 12 and hold the ear from slipping back under the scalp skin (see FIGS. 19 and 21). Correcting component 600 may, for example, be used for patients that are missing, or have a deformed, triangular fossa. The triangular fossa is the shallow depression in the anterior part of the top of the ear's auricle between the two crura (Crura of antihelix). The antihelix divides above and into the two crura forming the triangular fossa therebetween. In this embodiment, correcting component 600 is designed to fit into the area wherein the triangular fossa is normally located.

The correcting component 600 may also be used to correct cryptotia. In cryptotia, the root of the helix and a portion of the superior helix is under the scalp skin and needs to be manually pulled from that position and held in the correct position for a duration of time to correct the deformity.

As shown in FIGS. 18A and 18B, the correcting component 600 may include a main body 602 and first and second elongate members 604, 606 extending therefrom. Main body 602 may comprise a generally arcuate shape with a curved outer surface 608 configured for positioning underneath the upper portion of the helix 6 within the triangular fossa 12 (see FIG. 19). Elongate members 604, 606 define a channel or slit 610 therebetween. Slit 610 provides certain degree of flexibility to component 600 during placement to minimize trauma to the patient. In addition, elongate members 604, 606 may provide stability to main body 602 to help hold it in position within the fossa.

In some embodiments, the shape of correcting component 600 allows it to remain in place within the fossa 12 for a sufficient period of time to reshape the fossa 12 and correct the deformity. In other embodiments, one or more attachment components may be used to anchor the correcting component 600 in place. In one such embodiment, an adhesive (not shown), such as glue, surgical tape, or the like, is applied to the correcting component 600 to secure it within the fossa 12. In other embodiments, an additional correcting component and/or attachment component may be used.

FIGS. 20 and 21 illustrate one embodiment of an attachment component 620 that may be used with correcting component 600. As shown, attachment component 620 includes a base 622 coupled to an elongate member 624 that is, in turn, coupled to correcting component 600. Elongate member 624 is configured to extend around the helix 6 such that base 622 can be adhered to the patient's skin above the external ear (see FIG. 21).

While some of the correcting apparatuses of the present disclosure have been described thus far with embedded metal wires or filaments, the metal wires or filaments may be part of a metal mesh or a metal weave (not shown). The metal mesh or weave may be positioned at discrete locations on the main body of the correcting apparatus, or the metal mesh or weave may extend throughout the main body. Here, the metal wires or filaments form a metal mesh or weave that extends throughout the main body of the correcting apparatus.

Kits for correcting ear deformities in newborns and young infants can be provided which would include a set of correcting apparatuses similar to the ones shown and described herein, along with a user's guide. In one embodiment of a user's guide, the guide may take the form of a transparent template that may include an outline of a model human ear. The guide may be placed onto a photograph of a patient's ear. Using the line guides separating the various sections (e.g., A, B, C, and D) of the ear to be treated, the physician can identify which section(s) includes the deformity or deformities. The physician can then match the section(s) with the corresponding correcting apparatus(es) for that section(s). As a visual aid, these sections may also be colored for ease of use.

In an exemplary method of using the correcting apparatuses of the present disclosure, a physician could first select the appropriately sized and shaped correcting apparatus using the user's guide as described above, or by self-selecting the correcting apparatus from the set of apparatuses. Next, the physician can manipulate and bend the correcting apparatus to conform the shape of the correcting apparatus to a desired model ear shape. The conformed correcting apparatus can then be placed on the patient's ear for a time period to mold the patient's ear towards the desired model ear shape.

Although the correcting apparatuses and correction systems of the present disclosure are described herein for use in newborns and young infants, it is understood that the correcting apparatuses and correction systems may be equally applicable for use in older children as well as in adults who have external ear deformities. In addition, the correcting components disclosed herein may each be used alone, in combination with each other or in combination with other ear components. For example, conchal bowl component 200 may be used in combination with any of helical rim component 100, structural arch component 300, fossa component 400 and/or ear lobe component 500. Alternatively, structural arch component 300 may be used in combination with fossa component 400, ear lobe component 500 and/or helical rim component 100. In some embodiments, structural arch component 300 may be used as an additional attachment component for the other components.

Examples of other correcting components that may be used in combination with the features of the present disclosure can be found in commonly-assigned co-pending International Patent Application Nos. PCT/US19/60952 to CORRECTING APPARATUS FOR DEFORMED EXTERNAL EAR, filed Nov. 12, 2019 and PCT/US21/46349 to CORRECTING APPARATUS FOR DEFORMED EXTERNAL EAR, filed Aug. 17, 2021, the complete disclosures of which are hereby incorporated herein by reference for all purposes as if copied and pasted herein.

Correcting apparatuses and correction systems of the present disclosure may be customized to a patient using 3D printing techniques. For example, the dimension(s) of the model external ear may match the dimension(s) of the deformed external ear (such as in length, width, size, and/or density, among others). The components of the correcting apparatus (such as the main body) may also be printed with a three dimensional printer. The three dimensional printer may use the model external ear as the blueprint for the correcting apparatus. The three dimensional printer may match a shape and dimension(s) of the model external ear (e.g., such as a size) while printing the correcting apparatus.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

1-40. (canceled)

41. A correcting apparatus for remodeling a portion of an external ear, the apparatus comprising:
a correcting component having a main body shaped for insertion into, or in contact with, a conchal bowl of the ear and first and second projections extending anteriorly from the main body;
wherein the first projection is configured for positioning near, or in contact with, at least a portion of a cymba of the ear; and
wherein the second projection is configured to extend towards a tragus of the external ear.

42. The correcting apparatus of claim 41, further comprising a third projection.

43. The correcting apparatus of claim 41, wherein the main body comprises a material sufficiently rigid to remain in position during a time period of use sufficient to correct a deformity in the external ear.

44. The correcting apparatus of claim 41, wherein the main body comprises a first surface, a second opposing surface and a hole extending through the first and second surfaces for passing sound waves through the main body.

45. The correcting apparatus of claim 41, further comprising an attachment component configured for securing the correcting component against the surface of the external ear.

46. The correcting apparatus of claim 45, wherein the main body comprises an extended tab, and the attachment component comprises a slot for receiving the extended tab therethrough.

47. The correcting apparatus of claim 46, wherein the slot has a shape and size matching the extended tab of the main body.

48. The correcting apparatus of claim 45, wherein the attachment component has an opening to allow sound waves to pass therethrough.

49. The correcting apparatus of claim 45, wherein the attachment component comprises an adhesive.

50. A system for remodeling a portion of an external ear, the system comprising:
a first correcting component having a main body shaped for insertion into, or in contact with, a conchal bowl of the ear and first and second projections extending anteriorly from the main body, the first projection being configured for positioning near, or in contact with, at least a portion of a cymba of the ear, and the second projection being configured to extend towards a tragus of the external ear; and
a second correcting component having a main body configured for positioning between a surface of the external ear and a helix of the external ear.

51. The system of claim 50, wherein the first correcting component further comprises a third projection.

52. The system of claim 50, wherein the main body of the first correcting component and second component comprises a material sufficiently rigid to remain in position during a time period of use sufficient to correct a deformity in the external ear.

53. The system of claim 50, wherein the main body of the first correcting component comprises a first surface, a second opposing surface and a hole extending through the first and second surfaces for passing sound waves through the main body.

54. The system of claim 50, further comprising an attachment component configured for securing the first correcting component against the surface of the external ear.

55. The system of claim 54, wherein the main body of the first correcting component comprises an extended tab, and the attachment component comprises a slot for receiving the extended tab therethrough.

56. The system of claim 55, wherein the slot has a shape and size matching the extended tab of the main body of the first correcting component.

57. The system of claim 54, the attachment component has an opening to allow sound waves to pass therethrough.

58. The system of claim 54, wherein the attachment component comprises an adhesive.

59. The system of claim 50, further comprising a third correcting component configured for positioning within, or in contact with, a fossa triangularis of the external ear.

60. The system of claim 50, further comprising a third correcting component having a main body shaped for extending across a portion of the external ear, the main body comprises a first portion configured for contacting a helix of the internal ear and a second portion configured for contacting an anterior surface of the external ear.

61. The system of claim 60, wherein the third correcting component is configured to secure the first correcting component in position within the conchal bowl of the ear.

62-68. (canceled)
